(19) United States
(12) Patent Application Publication
(10) Pub. No.: US 2026/0174674 A1
(43) Pub. Date: Jun. 25, 2026

(54) INCOMPLETE AUTOPHAGY INDUCTION FOR THE TREATMENT OF CANCER

(71) Applicant: Northwestern University, Evanston, IL (US)

(72) Inventors: Maciej Lesniak, Evanston, IL (US); Jawad Fares, Evanston, IL (US)

(73) Assignee: Northwestern University, Evanston, IL (US)

(21) Appl. No.: 18/726,571

(22) PCT Filed: Jan. 3, 2023
(86) PCT No.: PCT/US2023/010018

§ 371 Date: Jul. 3, 2024

Related U.S. Application Data
(60) Provisional application No. 63/296,674, filed on Jan. 5, 2022.

(51) Int. Cl. A61K 9/00

(52) U.S. Cl. CPC A61K 9/0019; A61K 9/0014; A61K 9/0053

(57) ABSTRACT

Disclosed herein are methods and compositions for treating cancer using an incomplete autophagy inducer, such as metixene (aka. “methixene”), to trigger caspase-mediated apoptosis in cancer cells. The described methods and compositions can cause a significant decrease in tumor size and a significant prolongation in survival. The methods and compositions are useful for the treatment of cancers, such as brain cancer, breast cancer, lung cancer, and melanoma.

### CROSS REFERENCE TO RELATED APPLICATION

This application claims the benefit under 35 U.S.C. § 119(e) of U.S. Provisional Application No. 63/296,674, filed Jan. 5, 2022, the entire contents of which are incorporated herein by reference.

### STATEMENT OF GOVERNMENT INTEREST

This invention was made with government support under grants 5P50CA221747-03, 5R35CA197725-07, 5R01NS087990-05, and 5R01NS093903-04 awarded by the National Institutes of Health. The government has certain rights in the invention.

### TECHNICAL FIELD

The present invention relates generally to the field of cancer treatment. Disclosed herein are methods and compositions for treating cancer using an incomplete autophagy inducer, such as metixene (aka. “methixene”), to trigger caspase-mediated apoptosis in cancer cells. The described methods and compositions can cause a significant decrease in tumor size and a significant prolongation in survival. The methods and compositions are useful for the treatment of cancers such as brain cancer, breast cancer, lung cancer, and melanoma.

### BACKGROUND

Brain metastases are the most common type of central nervous system (CNS) malignancies. They often manifest with neurological impairment that portends poor quality of life and limits survival outcomes. It is estimated that 10-30% of all patients with cancer will develop brain metastases at one stage of the disease. The incidence of CNS spread may be increasing, though, as a result of improved diagnostics and better control of extracranial disease through systemic therapies.

Breast cancer is one of the major causes of brain metastases. It is the most common cancer among women, impacting 2.1 million per year. It is also the most common cause of cancer related deaths in women, with rates increasing in nearly every region globally. The incidence of brain metastases depends on the breast cancer molecular subtype, with human epidermal growth factor receptor 2 (HER2)-positive and triple-negative breast cancers having rates of brain metastases that can reach 50%. The introduction of anti-HER2 monoclonal antibody, trastuzumab, in HER2-positive breast cancer improved survival outcomes in systemic disease; however, the limited penetration of trastuzumab and other systemic drugs across the blood brain barrier (BBB) increase the incidence of brain metastases. This is thought to be due to the prolonged life span of patients with HER2-positive BCBM on treatment, and the ability of the BBB to shield penetrating cancer cells from therapeutic agents, allowing them to grow and colonize.

Metastasis is a hallmark of cancer that remains a primary cause of cancer-related deaths. A major limitation in treating patients with breast cancer brain metastases (BCBM) is the lack of clinical trials and therapeutic options. With more than 10,000 registered clinical trials for breast cancer, less than 1 percent include patients with brain metastases. Moreover, out of the trials that include patients with brain metastases, less than 15% are being completed, and less than 25% publish their results. As such, novel, effective agents against the disease that can be swiftly translated to the clinical scene are urgently needed.

### SUMMARY OF THE INVENTION

One embodiment relates to a method for treating a cancer (including a metastasized cancer) comprising administering to a subject in need of such treatment an effective amount of an agent that is capable of inducing incomplete autophagy in a cancer cell, including a primary cancer cell, a metastasized cancer cell, or a primary brain tumor cell.

In some embodiments, the agent is an antibody or a small molecule drug.

In some embodiments, the agent is a thioxanthene-containing small molecule wherein the thioxanthene comprises the following formula:

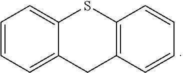 [SMILES: c1ccc2c(c1)Cc1ccccc1S2]

In some embodiments, the agent is a piperidine-containing small molecule wherein the piperidine comprises the following formula:

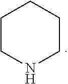

In some embodiments, the agent is metixene (aka. “methixene”) with the following chemical formula:

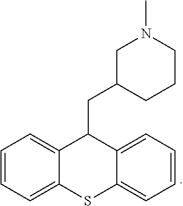 [SMILES: CN1CCCC(CC2c3ccccc3Sc3ccccc32)C1]

In some embodiments, the agent is metixene hydrochloride. In some embodiments, the agent is metixene hydrochloride hydrate.

In some embodiments, the metastasized cancer is a cancer that has metastasized to the brain. In some embodiments, the cancer that has metastasized to the brain is a breast cancer brain metastasis (BCBM), a lung cancer brain metastasis (LCBM) or a melanoma brain metastasis (MBM).

In some embodiments, the BCBM is from a HER-2 positive breast cancer. In some embodiments, BCBM is from a trastuzumab-resistant breast cancer. In some embodiments, the BCBM is from a triple-negative breast cancer.

In some embodiments, the agent is administered systemically. In some embodiments, the agent is administered locally.

In some embodiments, the agent is administered in combination with a chemotherapeutic agent or with radiotherapy.

In some embodiments, the effective amount of the agent is between about 0.05 mg/kg and 100 mg/kg.

In some embodiments, the agent is administered intermittently. In some embodiments, the agent is administered continuously.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D. CNS drug screen identifies metixene as an anti-cancer agent. (FIG. 1A) Cellular viability of BT-474Br and MDA-MB-231Br cells under treatment with different concentrations of metixene for 24 h and 48 h. (FIG. 1B) Caspase 3/7 activity in BT-474Br and MDA-MB-231Br cells under treatment with different concentrations of metixene for 24 h and 48 h. (FIG. 1C) Protein expression of cleaved caspase-3 after metixene treatment of BT-474Br cells. (FIG. 1D) Protein expression of cleaved caspase-3 after metixene treatment of MDA-MB-231Br cells.

FIGS. 2A-2E. Metixene decreases tumor size and prolongs survival. (FIG. 2A) Metixene decreases the size of mammary fat pad tumors. (FIG. 2B) Tumor weight decreased significantly at 0.1 mg/kg (P=0.0043) and 1.0 mg/kg (P=0.0004) of metixene treatment. (FIG. 2C) Tumor volume decreased significantly with metixene treatment at the given doses (p<0.0001). (FIG. 2D) Metixene prolongs survival significantly in intracranial models of BCBM (p=0.0008). Metixene increased median survival significantly from 52 days in controls to 64 days in treated groups. (FIG. 2E) Metixene prolongs survival significantly in intracarotid models of BCBM, with multiple brain metastases (p=0.037). Metixene increased median survival significantly from 44 days in controls to 67 days in treated groups.

FIGS. 3A-3J. Metixene induces NDRG-1 mediated incomplete autophagy in cancer cells. (FIG. 3A) Heat map of protein phosphorylation changes that were significant with a p<0.001 at 12 h. (FIG. 3B) Pathway analysis of changes in protein phosphorylation that were significant with a P<0.05 using the KEGG 2019 Human database. (FIG. 3C) LC3 protein expression analysis upon metixene treatment (10 μM) in MDA-MB-231Br cells at the specified time points. (FIG. 3D) Autophagy flux protein analysis in MDA-MB-231Br cells treated with metixene (M, 10 μM) and/or chloroquine (CQ, 25 μM) at 1 hour. (FIG. 3E) Representative LC3 puncta immunofluorescence in BT-474Br cells under metixene (10 μM) treatment. (FIG. 3F) LC3 protein expression in MDA-MB-231Br cells treated with increasing concentrations of metixene (μM). (FIG. 3G) Metixene induces NDRG1 expression and its phosphorylated form pNDRG1 significantly in a dose-dependent manner in MDA-MB-231Br cells. (FIG. 3H) CRISPR Cas9 knockout of NDRG1 led to autophagy completion. NDRG1 expression and phosphorylation regulate metixene-induced incomplete autophagy. (FIG. 3I) NDRG1 KO cells are significantly more viable than control cells after 10 μM (P=0.0271) and 20 μM (P<0.0001) metixene treatment. (FIG. 3J) NDRG1 knockout led to the reversal of the metixene apoptotic effect. Apoptosis is significantly reduced in the NDRG1 KO cells after 10 μM (P=0.0078) and 15 μM (P<0.0001) metixene treatment.

FIGS. 4A-4C. Metixene induces cell death in primary brain tumors and other metastatic cancers. (FIG. 4A) CT2A glioma cells were treated with increasing amount of metixene. Metixene showed potent cell-killing effect beginning at 5 μM. (FIG. 4B) H2030Br lung cancer brain metastatic cells were treated with increasing amount of metixene. Metixene showed potent cell-killing effect beginning at 10 μM. (FIG. 4C) WM3734 melanoma brain metastatic cells were treated with increasing amount of metixene. Metixene showed potent cell-killing effect beginning at 10 μM.

### DETAILED DESCRIPTION

### Definitions

Unless otherwise specified, “a” or “an” means “one or more.”

As used in this disclosure, the term “about” refers to a variation within approximately ±10% from a given value.

“Oral” or “peroral” administration refers to the introduction of a substance into a subject's body through or by way of the mouth and involves swallowing or transport through the oral mucosa (e.g., sublingual or buccal absorption) or both.

“Oronasal” administration refers to the introduction of a substance into a subject's body through or by way of the nose and the mouth, as would occur, for example, by placing one or more droplets in the nose. Oronasal administration involves transport processes associated with oral and intranasal administration.

“Parenteral administration” refers to the introduction of a substance into a subject's body through or by way of a route that does not include the digestive tract. Parenteral administration includes subcutaneous administration, intramuscular administration, transcutaneous administration, intradermal administration, intraperitoneal administration, intraocular administration, and intravenous administration.

“Topical administration” means the direct contact of a substance with tissue, such as skin or membrane, particularly the oral or buccal mucosa.

As used in this disclosure, the phrase “small molecule compound” refers to small organic chemical compound, generally having a molecular weight of less than 2000 daltons, 1500 daltons, 1000 daltons, 800 daltons, or 600 daltons.

The term “subject” encompasses human or non-human animal such as a companion animal, livestock animal or captured wild animal. In some embodiments, the subject is a subject who has cancer. In some embodiments, the subject is a subject who has metastasized cancer. In some embodiments, the subject is a subject who has cancer metastasized to the subject's brain. In some embodiments, the subject is a subject who has a primary brain tumor. In some embodiments, the subject is an adult, and in other embodiments, the subject is a child.

### Methods for Treating Cancer

An aspect of this disclosure is directed to a method for treating a cancer comprising administering to a subject in need of such treatment an effective amount of an agent is capable of inducing incomplete autophagy in a metastasized cancer cell or a primary brain tumor.

In some embodiments, the agent is an antibody or a small molecule drug.

In some embodiments, wherein the agent is a thioxanthene-containing small molecule wherein the thioxanthene contains the following formula:

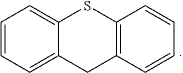 [SMILES: c1ccc2c(c1)Cc1ccccc1S2]

In some embodiments, the agent is a piperidine-containing small molecule wherein the piperidine contains the following formula:

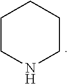

In some embodiments, the agent is metixene (aka. “methixene”) with the following chemical formula:

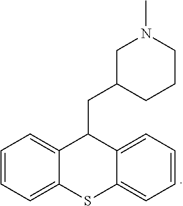 [SMILES: CN1CCCC(CC2c3ccccc3Sc3ccccc32)C1]

In some embodiments, the agent is metixene hydrochloride. In some embodiments, the agent is metixene hydrochloride hydrate.

In some embodiments, the metastasized cancer is a cancer that has metastasized to the brain. In some embodiments, the cancer that has metastasized to the brain is a breast cancer brain metastasis (BCBM), a lung cancer brain metastasis (LCBM) or a melanoma brain metastasis (MBM).

In some embodiments, the BCBM is from a HER-2 positive breast cancer. In some embodiments, BCBM is from a trastuzumab-resistant breast cancer. In some embodiments, the BCBM is from a triple-negative breast cancer.

In some embodiments, the cancer is a primary brain cancer. In some embodiments, the primary brain cancer is a glioma.

In some embodiments, the agent is administered systemically. In some embodiments, the agent is administered locally. In some embodiments, the agent is administered orally, oronasally, parenterally, or topically

In some embodiments, the agent is administered in combination with a chemotherapeutic agent or with radiotherapy.

In some embodiments, the effective amount of the agent is about 0.05 mg/kg, 0.1 mg/kg, 0.2 mg/kg, 0.5 mg/kg, 1 mg/kg, 8 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 150 mg/kg, 175 mg/kg or 200 mg/kg. In some embodiments, the effective amount of the agent is between about 0.05 mg/kg and about 100 mg/kg. In some embodiments, the effective amount of the agent is between about 0.05 mg/kg and about 10 mg/kg. In some embodiments, the effective amount of the agent is between about 1 mg/kg and about 20 mg/kg. In some embodiments, the effective amount of the agent is between about 5 mg/kg and about 20 mg/kg. In some embodiments, the effective amount of the agent is between about 10 mg/kg and about 50 mg/kg. In some embodiments, the effective amount of the agent is between about 20 mg/kg and about 100 mg/kg. In some embodiments, the effective amount of the agent is between about 40 mg/kg and about 150 mg/kg. In some embodiments, the effective amount of the agent is between about 50 mg/kg and about 200 mg/kg.

In some embodiments, the agent is administered intermittently. In some embodiments, the agent is administered continuously.

In some embodiments, administering or a grammatical variation thereof also refers to more than one doses with certain interval. In some embodiments, the interval is 1 hour, 2 hours, 6 hours, 12 hours, 1 day, 2 days, 3 days, 4 days, 5 days, 6 days, 1 week, 10 days, 2 weeks, 3 weeks, 1 month, 2 months, 3 months, 4 months, 5 months, 6 months, 1 year or longer. In some embodiments, one dose is repeated for once, twice, three times, four times, five times, six times, seven times, eight times, nine times, ten times or more. For example, cells as disclosed herein may be administered to a subject weekly and for up to four weeks. The compositions and therapies disclosed herein can be combined with other therapies, e.g., chemotherapy, CAR-NK or CAR-T cell therapy.

### Pharmaceutical Compositions Comprising an Agent Capable of Inducing Incomplete Autophagy in a Cancer Cell

In some embodiments, an agent capable of inducing incomplete autophagy in a cancer cell as defined herein can be combined with a pharmaceutically acceptable carrier prior to administration. For the purposes of this disclosure, “pharmaceutically acceptable carriers” means any of the standard pharmaceutical carriers. Examples of suitable carriers are well known in the art and may include, but are not limited to, any of the standard pharmaceutical carriers such as a phosphate buffered saline solution and various wetting agents. Other carriers may include additives used in tablets, granules and capsules, and the like. Typically such carriers contain excipients such as starch, milk, sugar, certain types of clay, gelatin, stearic acid or salts thereof, magnesium or calcium stearate, talc, vegetable fats or oils, gum, glycols or other known excipients. Such carriers may also include flavor and color additives or other ingredients. Compositions comprising such carriers are formulated by well-known conventional methods.

In some embodiments, wherein the agent is a thioxanthene-containing small molecule wherein the thioxanthene contains the following formula:

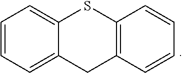 [SMILES: c1ccc2c(c1)Cc1ccccc1S2]

In some embodiments, the agent is a piperidine-containing small molecule wherein the piperidine contains the following formula:

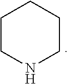

In some embodiments, the agent is metixene (aka. “methixene”) with the following chemical formula:

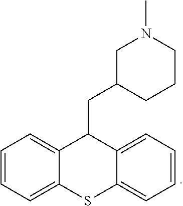 [SMILES: CN1CCCC(CC2c3ccccc3Sc3ccccc32)C1]

In some embodiments, the agent is metixene hydrochloride. In some embodiments, the agent is metixene hydrochloride hydrate.

An agent capable of inducing incomplete autophagy in a cancer cell can be admixed with a pharmaceutically acceptable carrier to make a pharmaceutical preparation in any conventional form including, inter alia, a solid form such as tablets, capsules (e.g. hard or soft gelatin capsules), pills, cachets, powders, granules, and the like; a liquid form such as solutions, suspensions; or in micronized powders, sprays, aerosols and the like.

The pharmaceutical compositions of the present disclosure can be used in liquid, solid, tablet, capsule, pill, ointment, cream, nebulized or other forms as explained below. In some embodiments, the composition of the present disclosure may be administered by different routes of administration such as oral, oronasal, parenteral or topical.

The pharmaceutical preparations of the present disclosure can be made up in any conventional form including, inter alia: (a) a solid form for oral administration such as tablets, capsules (e.g. hard or soft gelatin capsules), pills, sachets, powders, granules, and the like; (b) preparations for topical administrations such as solutions, suspensions, ointments, creams, gels, micronized powders, sprays, aerosols and the like. The pharmaceutical preparations may be sterilized and/or may contain adjuvants such as preservatives, stabilizers, wetting agents, emulsifiers, salts for varying the osmotic pressure and/or buffers.

For topical administration to the skin or mucous membrane the aforementioned composition is preferably prepared as ointments, tinctures, creams, gels, solution, lotions, sprays; aerosols and dry powder for inhalation, suspensions, shampoos, hair soaps, perfumes and the like. In fact, any conventional composition can be utilized in this invention. Among the preferred methods of applying the composition containing the agents of this invention is in the form of an ointment, gel, cream, lotion, spray; aerosol or dry powder for inhalation. The pharmaceutical preparation for topical administration to the skin can be prepared by mixing the aforementioned active ingredient with non-toxic, therapeutically inert, solid or liquid carriers customarily used in such preparation. These preparations generally contain 0.01 to 5.0 percent by weight, or 0.1 to 1.0 percent by weight, of the active ingredient, based on the total weight of the composition.

In preparing the topical preparations described above, additives such as preservatives, thickeners, perfumes and the like conventional in the art of pharmaceutical compounding of topical preparation can be used. In addition, conventional antioxidants or mixtures of conventional antioxidants can be incorporated into the topical preparations containing the aforementioned active agent. Among the conventional antioxidants which can be utilized in these preparations are included N-methyl-a-tocopherolamine, tocopherols, butylated hydroxyanisole, butylated hydroxytoluene, ethoxyquin and the like.

Cream-based pharmaceutical formulations containing the active agent, used in accordance with this invention, are composed of aqueous emulsions containing a fatty acid alcohol, semi-solid petroleum hydrocarbon, ethylene glycol and an emulsifying agent.

Ointment formulations containing the active agent in accordance with this invention comprise admixtures of a semi-solid petroleum hydrocarbon with a solvent dispersion of the active material. Cream compositions containing the active ingredient for use in this invention preferably comprise emulsions formed from a water phase of a humectant, a viscosity stabilizer and water, an oil phase of a fatty acid alcohol, a semi-solid petroleum hydrocarbon and an emulsifying agent and a phase containing the active agent dispersed in an aqueous stabilizer-buffer solution. Stabilizers may be added to the topical preparation. Any conventional stabilizer can be utilized in accordance with this invention. In the oil phase, fatty acid alcohol components function as a stabilizer. These fatty acid alcohol components function as a stabilizer. These fatty acid alcohol components are derived from the reduction of a long-chain saturated fatty acid containing at least-14 carbon atoms.

Also, conventional perfumes and lotions generally utilized in topical preparation for the hair can be utilized in accordance with this invention. Furthermore, if desired, conventional emulsifying agents can be utilized in the topical preparations of this invention.

In some embodiments, compositions comprising an agent capable of inducing incomplete autophagy in a cancer cell can be administered by aerosol. For example, this can be accomplished by preparing an aqueous aerosol, liposomal preparation or solid particles containing a composition comprising an agent capable of inducing incomplete autophagy in a cancer cell preparation. A nonaqueous (e.g., fluorocarbon propellant) suspension could be used. Sonic nebulizers can also be used. An aqueous aerosol is made by formulating an aqueous solution or suspension of the agent together with conventional pharmaceutically acceptable carriers and stabilizers. The carriers and stabilizers vary with the requirements of the particular compound.

### Use of an Agent Capable of Inducing Incomplete Autophagy in a Cancer Cell for the Manufacture of a Medicament.

Another aspect of this disclosure is directed to a use of an agent capable of inducing incomplete autophagy in a cancer cell for the manufacture of a medicament for use in treatment of cancer. In some embodiments, the cancer is a metastasized cancer. In some embodiments, the cancer is a cancer metastasized to the brain. In some embodiments, the cancer that has metastasized to the brain is a breast cancer brain metastasis (BCBM), a lung cancer brain metastasis (LCBM) or a melanoma brain metastasis (MBM). In some embodiments, the cancer is a primary brain cancer.

In some embodiments, the agent is an antibody or a small molecule drug.

In some embodiments, wherein the agent is a thioxanthene-containing small molecule wherein the thioxanthene contains the following formula:

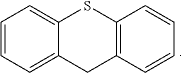 [SMILES: c1ccc2c(c1)Cc1ccccc1S2]

In some embodiments, the agent is a piperidine-containing small molecule wherein the piperidine contains the following formula:

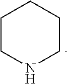

In some embodiments, the agent is metixene (aka. “methixene”) with the following chemical formula:

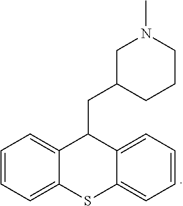 [SMILES: CN1CCCC(CC2c3ccccc3Sc3ccccc32)C1]

In some embodiments, the agent is metixene hydrochloride. In some embodiments, the agent is metixene hydrochloride hydrate.

In some embodiments, the metastasized cancer is a cancer that has metastasized to the brain. In some embodiments, the cancer that has metastasized to the brain is a breast cancer brain metastasis (BCBM), a lung cancer brain metastasis (LCBM) or a melanoma brain metastasis (MBM).

In some embodiments, the BCBM is from a HER-2 positive breast cancer. In some embodiments, BCBM is from a trastuzumab-resistant breast cancer. In some embodiments, the BCBM is from a triple-negative breast cancer.

In some embodiments, the agent is administered systemically. In some embodiments, the agent is administered locally. In some embodiments, the agent is administered orally, oronasally, parenterally, or topically.

In some embodiments, the agent is administered in combination with a chemotherapeutic agent or with radiotherapy.

In some embodiments, the effective amount of the agent is about 0.05 mg/kg, 0.1 mg/kg, 0.2 mg/kg, 0.5 mg/kg, 1 mg/kg, 8 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 150 mg/kg, 175 mg/kg or 200 mg/kg. In some embodiments, the effective amount of the agent is between about 0.05 mg/kg and 100 mg/kg. In some embodiments, the effective amount of the agent is between about 0.05 mg/kg and about 10 mg/kg. In some embodiments, the effective amount of the agent is between about 1 mg/kg and about 20 mg/kg. In some embodiments, the effective amount of the agent is between about 5 mg/kg and about 20 mg/kg. In some embodiments, the effective amount of the agent is between about 10 mg/kg and about 50 mg/kg. In some embodiments, the effective amount of the agent is between about 20 mg/kg and about 100 mg/kg. In some embodiments, the effective amount of the agent is between about 40 mg/kg and about 150 mg/kg. In some embodiments, the effective amount of the agent is between about 50 mg/kg and about 200 mg/kg.

In some embodiments, the agent is administered intermittently. In some embodiments, the agent is administered continuously.

In some embodiments, administering or a grammatical variation thereof also refers to more than one doses with certain interval. In some embodiments, the interval is 1 hour, 2 hours, 6 hours, 12 hours, 1 day, 2 days, 3 days, 4 days, 5 days, 6 days, 1 week, 10 days, 2 weeks, 3 weeks, 1 month, 2 months, 3 months, 4 months, 5 months, 6 months, 1 year or longer. In some embodiments, one dose is repeated for once, twice, three times, four times, five times, six times, seven times, eight times, nine times, ten times or more. For example, cells as disclosed herein may be administered to a subject weekly and for up to four weeks. The compositions and therapies disclosed herein can be combined with other therapies, e.g., chemotherapy, CAR-NK or CAR-T cell therapy.

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The specific examples listed below are only illustrative and by no means limiting.

### EXAMPLES

### Example 1: Materials and Methods

### Central Nervous System (CNS) Drug Screening

The Prestwick CNS Drug Library of 320 CNS compounds was purchased from Prestwick Chemical Libraries for screening. Cells were seeded at a density of 5000 cells/well in clear, flatbottomed, black-walled, 96-well plates. Each compound from the library was added at a final concentration of 25 μM per well 1 day after seeding of cells. After three days of treatment, cell viability was measured using CellTiter-Glo® Luminescent Cell Viability Assay (Promega) according to the manufacturer's instructions.

### Animal Experiments

All animal studies were completed per the National Institutes of Health guidelines on the care and use of laboratory animals for research purposes. The Institutional Committee on Animal Use at Northwestern University approved the protocols. Six- to eight-week-old athymic, immunodeficient (nu/nu) female mice were obtained from Charles River and maintained in a specific pathogen-free facility. For mammary gland injections, 1×10⁶ HCC1954 cells were injected in the inguinal mammary fat pad of nude female mice. For the stereotactic intracranial injection, mice were placed in a stereotactic frame, and 5×10⁵ BT-474Br cells were intracranially injected at a depth of 3 mm. For intracarotid artery injections, 1×10⁶ MDA-MB-231Br cells were injected using a 31-gauge BD insulin syringe into the lumen of internal carotid artery.

### Reverse Phase Protein Array

A reverse-phase protein array (RPPA) was performed in the MD Anderson Cancer Center (MDACC) comprehensive cancer center support grant (CCSG) core as described at MD Anderson education and research website.

### Statistical Analysis

Data were analyzed using a two-tailed t test or one-way analysis of variance with multiple comparisons, as stated in the figure legends (statistical significance cutoff=0.05). Error bars represent SDs. Statistical significance in the survival study was determined via a two-sided logrank test using GraphPad Prism 8.

### Example 2: CNS Drug Screening Identifies Agents with Anti-Cancer Effects in Brain Metastases

A blinded screen was conducted on a drug library comprising 320 structurally diverse drugs that are FDA approved, blood brain barrier (BBB) permeable, and known for their pharmacological effects on the CNS to identify potential hits for therapeutic intervention against brain metastases. Using BT-474Br, a HER2-positive BCBM cell line, the efficacy of the 320 compounds were screened at a standard concentration of 25 μM. After 72 hours of treatment, a cell viability assay was performed using Cell Titer Glo to determine the number of viable, metabolically active cells in culture. The screen yielded five different therapeutic agents that caused a vast reduction (>95%) in cell viability in vitro.

HER2-positive breast cancer has the greatest risk for intracranial-specific metastases. When metastatic cancer cells colonize the brain, they develop resistance to systemic therapies, such as trastuzumab. For this reason, another blinded screen was conducted on a metastatic breast cancer cell line that is trastuzumab-resistant, HCC1954. At 25 μM, the screen yielded five hits that caused a nearly complete loss of cell viability. Interestingly, metixene, an antiparkinsonian agent, was a top hit in both screens. Next, the effect of metixene on different metastatic breast cancer cell lines was determined by measuring its IC50 in two HER2-positive cell lines (BT474 and HCC1954) and five triple-negative breast cancer cell lines (MDAMB-231-Br, HCC1806, HS578T, HCC3153, and SUM159) after 72 hours of treatment. Metixene was shown to be a potent inhibitor of BCBM regardless of subtype.

### Example 3: Metixene Induces Caspase-Mediated Cell Death

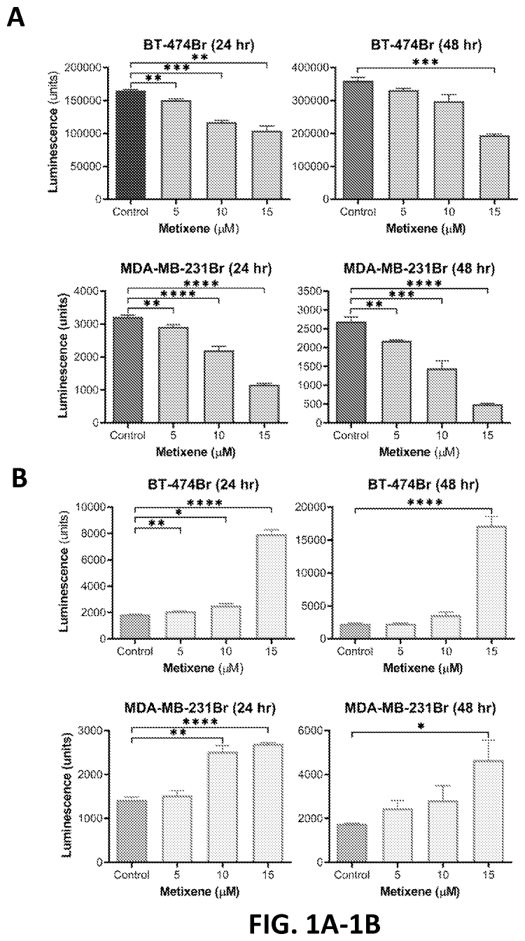

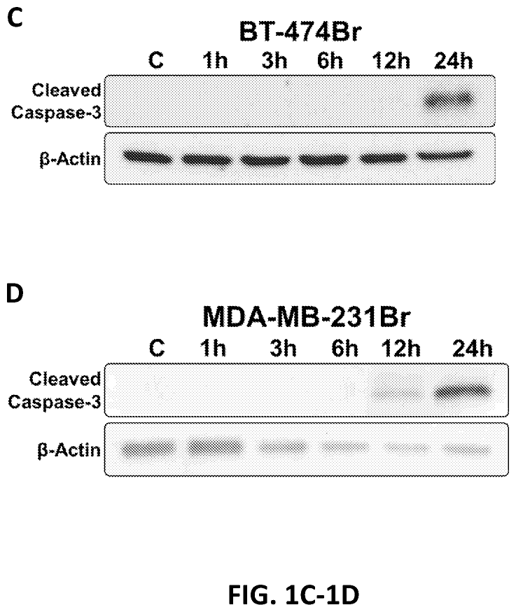

Following the determination of an appropriate range for treatment of BCBM cells, the anti-cancer activity of metixene was evaluated in the brain metastatic HER2-positive BT-474Br and the triple-negative MDA-MB-231Br cell lines, using increasing concentrations of the drug at different time points. After 24 hours of treatment, cell viability assays revealed a significant reduction in cell viability in the two cell lines. After 48 hours of treatment, cell viability significantly decreased at 15 μM in BT-474Br and at 5, 10, and 15 μM in MDA-MB-231Br (FIG. 1A).

Next, whether metixene induces apoptotic cell death was investigated. After 24 hours of treatment, caspase 3/7 assays showed a significant increase in caspase 3/7 activity in BT-474Br and at 10 μM and 15 μM in MDA-MB-231Br. After 48 hours, caspase 3/7 activity increased significantly at 15 μM in BT-474Br and MDA-MB-231Br (FIG. 1B). It was further shown that cleaved caspase-3 protein levels markedly increase after 24 hours of treatment in the two cell lines, BT-474Br and MDA-MB-231Br (FIGS. 1C and 1D). Immunofluorescence analysis of metixene-treated BT-474Br and MDA-MB-231Br cells confirmed increased cleaved caspase-3 expression.

### Example 4: Metixene Decreases Tumor Size In Vivo in Mice Bearing Metastatic Breast Cancer

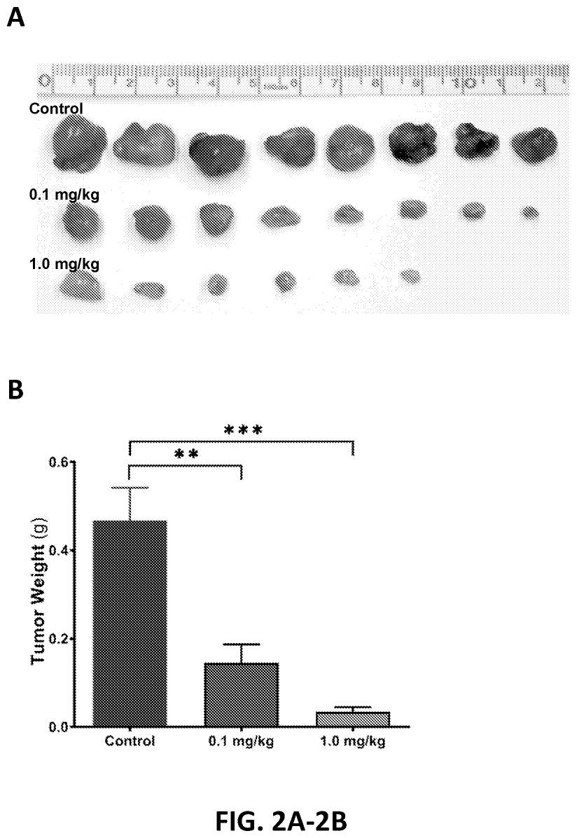

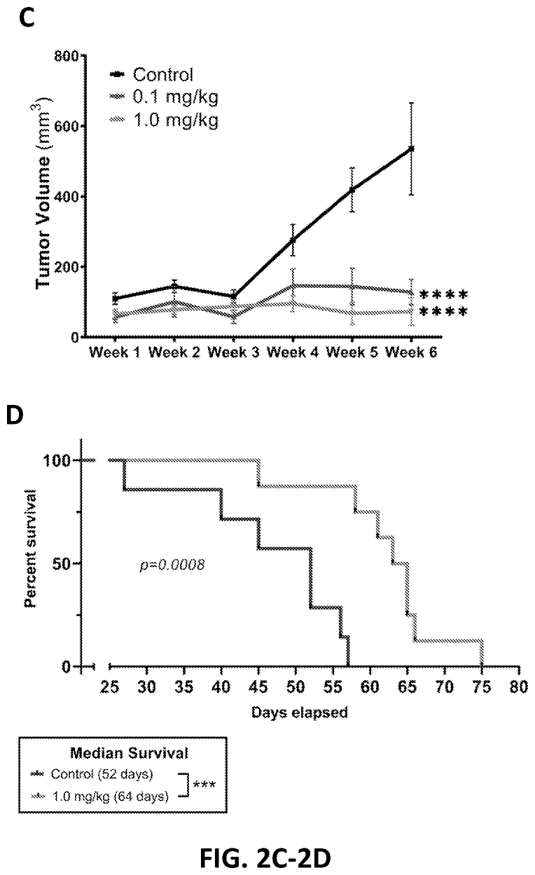

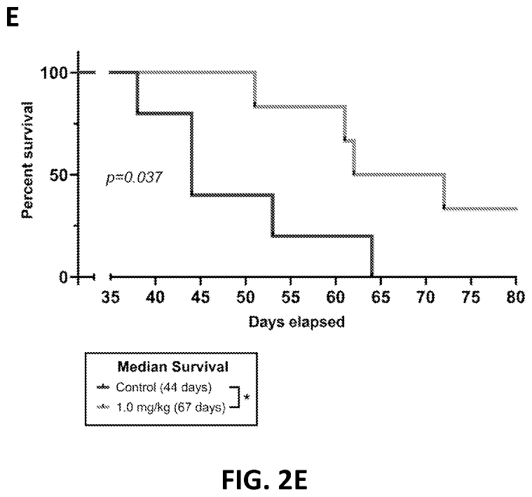

The efficacy of metixene hydrochloride was evaluated using an in vivo model. HCC1954 cells were injected into the mammary fat pads of nude mice. After the formation and establishment of 5 mm breast tumors, mice were randomly divided into three groups whereby treatment was administered intraperitoneally: control (25% captisol); metixene (0.1 mg/kg); and metixene (1.0 mg/kg). The dosages were given three times a week. After 6 weeks, fat pad tumors were collected and analyzed (FIG. 2A). Tumor weight and volume were significantly decreased (p<0.001) in the groups receiving metixene at the two doses, respectively (FIGS. 2B and 2C). Cleaved caspase-3 staining of tumor sections showed a significant increase in percent of cleaved caspase-3-positive cells in metixene-treated groups at 0.1 mg/kg (p=0.001) and 1.0 mg/kg (p=0.0002).

### Example 5: Metixene Improves Survival In Vivo in Different Preclinical Models of Brain Metastases

After showing its anti-tumor effect in vivo, whether metixene could improve survival in different preclinical models of brain metastases was tested. A stereotactic intracranial injection of HER2-positive BT474-Br cells was performed in nude mice. After 10 days of recovery and tumor growth, the mice were divided into two groups that were treated intraperitoneally: control (25% captisol, n=7); and metixene (1.0 mg/kg, n=8). The dosages were given three times per week. The median survival of control mice was 52 days; however, metixene treated mice showed a median survival of 64 days (FIG. 2D). This shows that metixene significantly improved survival in intracranial models of brain metastases (p=0.0008). Supporting this observation, the measurement of luciferase signal in the brain showed a significant reduction in the metixene treated group as compared to controls after six weeks of treatment (p=0.02).

These results were further validated in a model of multiple brain metastases. The model was established through a systemic intracarotid injection of triple-negative MDA-MB-231-Br cells in nude mice. Ten days after injection of tumor cells, mice were randomly divided into two groups and treated intraperitoneally with: control (25% captisol, n=5) and metixene (1.0 mg/kg, n=6). The treatments were given three times per week. In comparison to controls that exhibited a median survival of 44 days, metixene-treated mice showed a median survival of 67 days. Therefore, metixene significantly increased survival by 52% in comparison to controls (p=0.037) (FIG. 2E). Histological examination of the brains harvested from these mice showed multiple metastases, vascular co-option, and micrometastases formation. Therefore, metixene was capable of increasing median survival significantly in two preclinical models of BCBM.

### Example 6: Metixene Activates Macroautophagy Signaling Pathways and Induces Incomplete Autophagy

The inventors then set out to determine the mechanism of action of metixene, an agent with known antimuscarinic and antihistaminic properties. The expression of muscarinic and histaminic receptors that are present in the CNS were checked to see if their expression on different breast cancer cell lines correlates with their IC50 results when treated with metixene. The results determined that the RNA levels for muscarinic (M1-M5) or histaminic (H3) receptors did not correlate with the IC50 data relating to the breast cancer cell lines. This indicated that metixene's anti-cancer activity was not mediated through muscarinic or histaminic receptors.

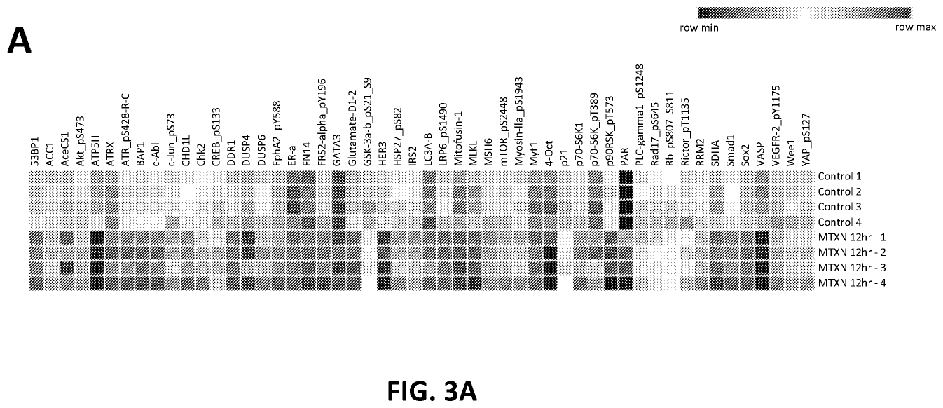

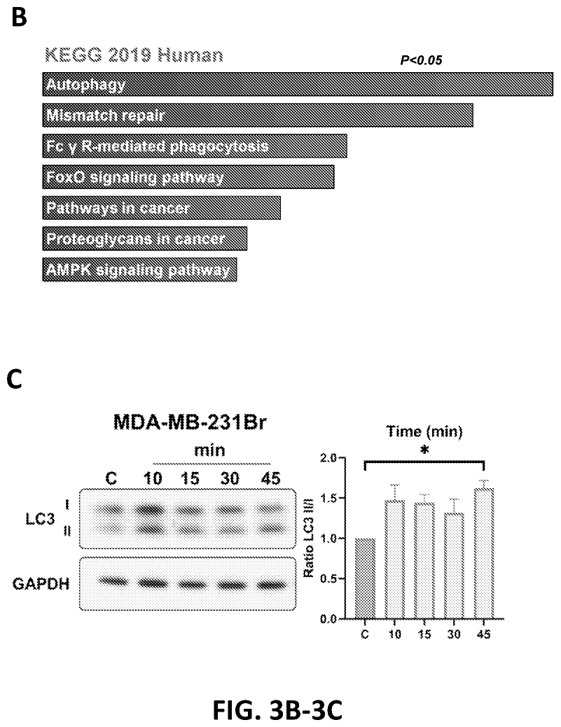

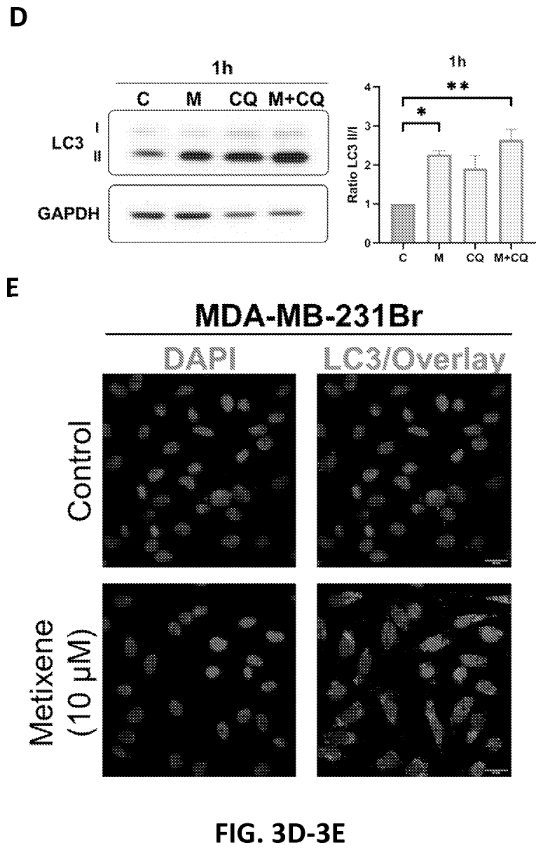

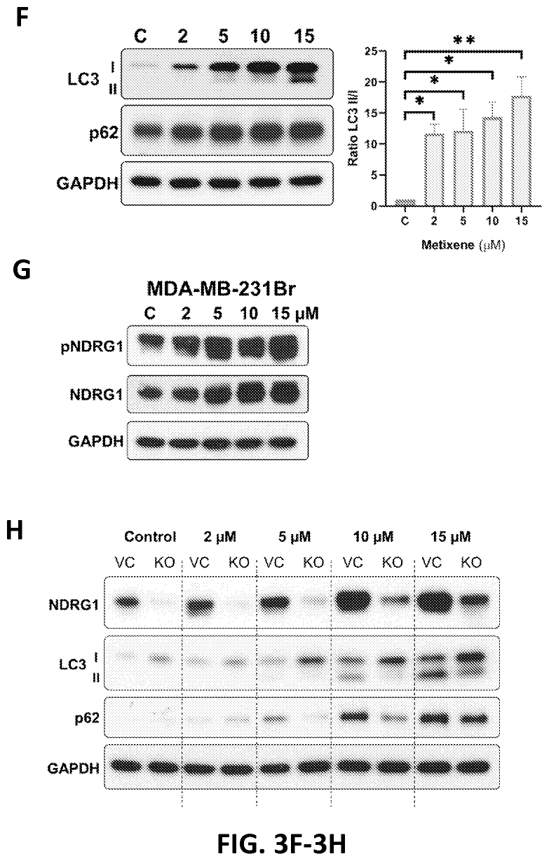

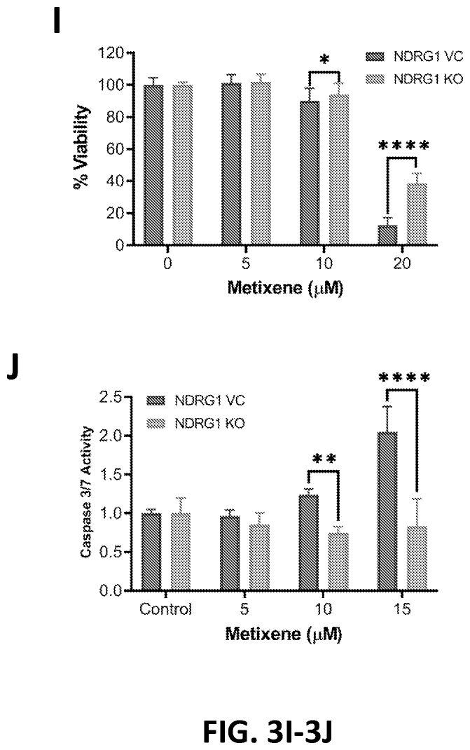

An exploratory RPPA was conducted to identify adaptive responses that mediate the anticancer activity of metixene in a BCBM cell line. BT474-Br cells were treated with 10 μM metixene hydrochloride for 12 and 24 hours, and RPPA was conducted to assess changes in the expression of different signaling pathways. Four biological replicates for each condition (control, 12 hours, and 24 hours) were analyzed. Analysis of protein expression and phosphorylation showed autophagy as a primary biological mechanism regulated by metixene in comparison to controls (P<0.05). Next, proteins that exhibit significant changes in expression or phosphorylation with a p-value less than 0.001 were selected. Results showed that a total of 52 and 26 proteins exhibited a significant alteration in their expression or phosphorylation status after 12 and 24 hours of treatment, respectively (FIG. 3A). The selected proteins helped to identify the signaling pathways that were involved in response to metixene. At both 12 and 24 hours, marked activation of multiple components of the MAPK, PI3K/Akt, apoptosis and autophagy, and mTOR signaling pathways were observed. These pathways are importantly implicated in macroautophagy, demonstrating that metixene is a vital regulator of the process in BCBM (FIG. 3B).

### Example 7: Metixene Induces Autophagy Signaling in Primary and Metastatic Breast Cancer Cells

Next, MDA-MB-231-Br cells were treated with metixene for different durations to determine the mechanism of autophagic signaling in brain metastatic cells. Conversion of LC3 I to LC3 II was shown to increase as early as ten minutes after metixene treatment (FIG. 3C). Afterward, autophagy flux changes after ten minutes, one hour, and three hours of treatment were tested. LC3 II levels in control (untreated) cells, cells treated with chloroquine (25 μM), cells treated with metixene (10 μM), and cells treated with metixene and chloroquine were compared. Cells treated with the combination of metixene and chloroquine had higher LC3 II levels than that of untreated cells or cells treated with either agent alone (FIG. 3D). This data indicates that autophagy signaling is being induced by metixene treatment.

The efficacy of metixene in inducing autophagy was tested in two primary breast and brain metastatic cancer cell lines, using BT-474 and BT-474Br and MDA-MB-231 and MDA-MB-231Br, respectively. LC3 II levels increased as the concentration of metixene increased. In addition the ratio of LC3 II to I levels trended upwards with increasing concentration, demonstrating autophagic induction (FIG. 3D). Furthermore, immunofluorescence staining of LC3 showed a significant increase in the accumulation of puncta in metixene-treated BT-474Br cells (P=0.015) (FIG. 3E). Altogether, this indicates that metixene hydrochloride is inducing autophagy signaling in cancer cells. Nonetheless, SQSTM1/p62 levels increased with increasing concentration (FIG. 3F), indicating that the cargo carrier is not getting degraded and that the autophagic process is incomplete.

### Example 8: NDRG1 Regulates Metixene-Induced Incomplete Autophagy and Caspase-Mediated Apoptosis

Upon further analysis of the RPPA data, the phosphorylation of N-Myc Downstream Regulated 1 (NDRG1) was noted to be relevant in four major pathways related to autophagy after 24 hours of metixene treatment. The expression of NDRG1 and its phosphorylated form, pNDRG1, increased significantly in a dose-dependent manner in MDA-MB-231Br after treatment with metixene (FIG. 3G). To confirm the role of NDRG1 in autophagy, NDRG1 knockout (KO) cells were generated using CRISPR Cas9 in MDA-MB-231Br cells. The cells were then treated with metixene in a dose-dependent manner and compared to cells transfected with a vector control. Western blots showed that LC3II levels decreased significantly in NDRG1 KO cells. Furthermore, the significant decrease in p62 levels in NDRG1 KO cells indicated that autophagy is being completed in comparison to NDRG1 VC cells (FIG. 3H). This showed that NDRG1 expression and phosphorylation regulate metixene-induced autophagy.

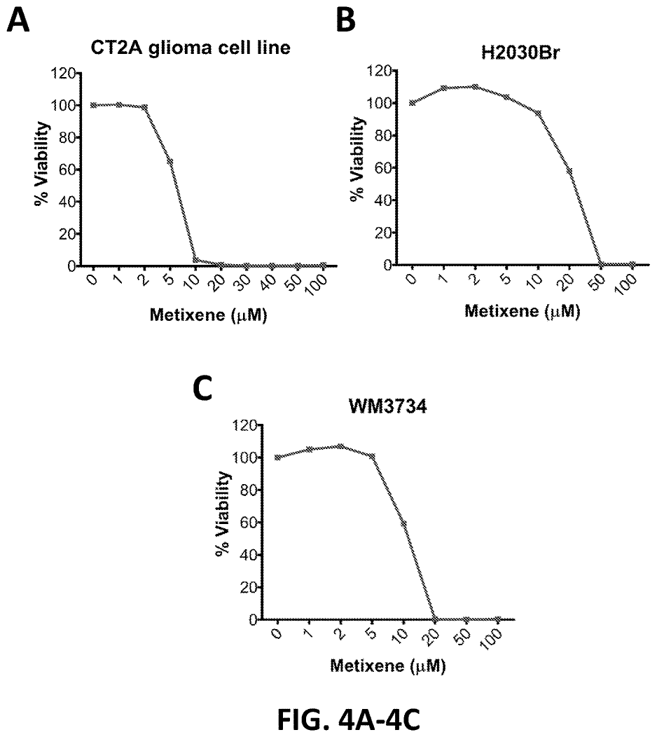

The inventors then checked whether NDRG1 is further involved in the apoptotic cell death induced by metixene. A cell viability assay was conducted to check the effect of NDRG1 KO versus vector control upon metixene dose-dependent treatment. Results showed that NDRG1 KO cells were significantly more viable than control cells at 10 μM (P=0.0271) and 20 μM (P<0.0001) (FIG. 3I). A caspase 3/7 assay further confirmed that apoptosis was significantly reduced in the NDRG1 KO cells at 10 μM (P=0.0078) and 15 μM (P<0.0001) (FIG. 3J). Altogether, this shows that metixene induces incomplete autophagy and apoptosis through NDRG1 expression and phosphorylation. Upon NDRG1 KO, autophagy is completed, and the caspase-mediated pathway of apoptosis is not activated. This mechanism applies to primary brain cancers, such as glioblastoma (FIG. 4A), and other metastatic cancers to the brain, such as lung cancer (FIG. 4B) and melanoma (FIG. 4C).

### Example 9

Metastasis is a hallmark of cancer that involves complex processes to establish tumors in distant organs. A lot of efforts have been aimed to identify therapeutic agents that can be effective against metastatic cancer; however, literature on the treatment of preexisting brain metastases, specifically, remains scarce. In this study, the inventors conducted a premier, blinded CNS drug screen of BBB permeable and FDA approved agents against BCBM cell lines. Metixene showed efficacy against BCBM cells in vitro and in vivo, as it prolonged survival significantly in preclinical models of brain metastases, regardless of tumor subtype. Mechanistic exploration revealed differences in autophagy signaling in tumor cells treated with metixene.

Drug repositioning or repurposing in cancer is a strategy that aims to reuse existing medical agents developed for other diseases as anti-cancer treatments. In brain metastases, novel anti-cancer therapies must have the ability to cross the BBB and to instigate cytotoxic effects against metastatic cancer cells that are often resistant to standard therapies. As such, the inventors conducted a screening on trastuzumab-sensitive and resistant brain metastatic cells using a library of agents that are known to cross the BBB. The instant screen identified a number of agents that drastically impaired cell viability in two different BCBM cell lines and provided the opportunity to pursue further translational research in brain metastases using the top identified agents.

Metixene was selected as a novel anti-tumorigenic agent for investigation. Metixene is a tertiary antimuscarinic, anticholinergic agent. It also exhibits antihistaminic and direct antispasmodic properties. It is FDA approved and used for the symptomatic treatment of Parkinsonism, including the alleviation of the extrapyramidal syndrome, and can be used as an adjunct for gastrointestinal hypermobility. Its route of administration is oral and the usual dosage for adults is 1 or 2 mg three times daily. Metixene has few reported side effects; it can dilate the pupils and inhibit the salivary secretions but to a lesser degree than does atropine. Metixene has not been previously reported to have anti-cancer activity.

Metixene was found to be a modulator of autophagy signaling in BCBM. Recent genetic studies and emerging functional work show that similar and overlapping pathways are involved in both Parkinson's disease and cancer. Parkinson's disease is a protein-misfolding disease, where misfolded α-synuclein aggregates and accumulates in Lewy bodies, leading to neurodegeneration. In cancer, protein misfolding and aggregation can activate oncoproteins or inactivate tumor suppressors, leading to tumorigenesis. As such, without being bound to a particular theory, it is believed that the autophagy-lysosomal pathway can help degrade protein aggregates and organelles by autophagosome engulfment and fusion to lysosomes that contain hydrolases. Modulators of this pathway, such as 17-AAG and rapamycin, have been shown to be effective in reducing α-synuclein in cells. Furthermore, autophagy regulation in animal models has been shown to suppress tumor formation. Temsirolimus, a rapamycin derivative, was approved for the treatment of renal cell carcinoma.

Autophagy modulators have been used in the setting of brain metastases. The addition of chloroquine to whole-brain radiation significantly improved median progression-free survival (NR vs 13.5 months, p=0.008) and decreased mortality related to brain disease progression (NR vs 25.6 months, p=0.016) in comparison to controls, with no increase in toxicity. Nevertheless, it did not control extracranial disease and did not significantly increase overall survival (10.2 vs 7.42 months, P=0.839). A major limitation of autophagy modulators, such as chloroquine, in brain tumor settings is their low permeability of the BBB. This necessitates high concentrations to achieve the desired autophagic effects, which can become considerably toxic. As such, metixene—a CNS agent that is BBB permeable, FDA approved with a low toxicity profile—can be a promising therapeutic option in clinical settings of BCBM, as it has shown efficacy against primary and brain metastatic breast cancer cells in vitro and in vivo.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention. All of the publications, patent applications and patents cited in this specification are incorporated herein by reference in their entirety.

1. A method for treating a cancer comprising administering to a subject in need of such treatment an effective amount of an agent that is capable of inducing incomplete autophagy in a metastasized cancer cell or a primary brain tumor cell.

2. The method of claim 1, wherein the agent is an antibody or a drug.

3. The method of claim 1, wherein the agent is a thioxanthene-containing drug wherein the thioxanthene comprises a moiety including the following structure:

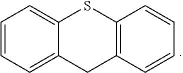 [SMILES: c1ccc2c(c1)Cc1ccccc1S2]

4. The method of claim 1, wherein the agent is a piperidine-containing small molecule wherein the piperidine contains the following formula:

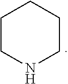

5. The method of claim 3, wherein the agent is metixene with the following chemical formula:

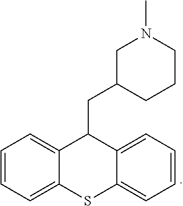 [SMILES: CN1CCCC(CC2c3ccccc3Sc3ccccc32)C1]

6. The method of claim 5, wherein the agent is metixene hydrochloride.

7. The method of claim 5, wherein the agent is metixene hydrochloride hydrate.

8. The method of claim 1, wherein the metastasized cancer is a cancer that has metastasized to the brain.

9. The method of claim 8, wherein the cancer that has metastasized to the brain is a breast cancer brain metastasis (BCBM), a lung cancer brain metastasis (LCBM) or a melanoma brain metastasis (MBM).

10. The method of claim 9, wherein the BCBM is from a HER-2 positive breast cancer.

11. The method of claim 9, wherein the BCBM is from a trastuzumab-resistant breast cancer.

12. The method of claim 9, wherein the BCBM is from a triple-negative breast cancer.

13. The method of claim 1, wherein the cancer is a primary brain cancer.

14. The method of claim 1, wherein the agent is administered systemically.

15. The method of claim 1, wherein the agent is administered locally.

16. The method of claim 1, wherein the agent is administered in combination with a chemotherapeutic agent or with radiotherapy.

17. The method of claim 1, wherein the effective amount of the agent is between about 0.05 mg/kg and 100 mg/kg.

18. The method of claim 1, wherein the agent is administered intermittently.

19. The method of claim 1, wherein the agent is administered continuously.
